(19) United States
(12) Patent Application Publication
(10) Pub. No.: US 2025/0135280 A1
(43) Pub. Date: May 1, 2025

(54) SYSTEM AND METHOD FOR TRACKING AND MEASURING BODY FLEXIBILITY AND BALANCE DURING PHYSICAL ACTIVITY

(71) Applicant: Samsung Electronics Co., Ltd., Suwon-si (KR)

(72) Inventors: Gunjan GUPTA, Noida (IN); Himanshu DIDDEN, Noida (IN); Akshay BINDLISH, Noida (IN); Anil KUMAR, Noida (IN); Anand Kumar ASATI, Noida (IN)

(21) Appl. No.: 18/793,189

(22) Filed: Aug. 2, 2024

Related U.S. Application Data
(63) Continuation of application No. PCT/KR2024/010835, filed on Jul. 25, 2024.

(30) Foreign Application Priority Data
Oct. 26, 2023 (IN) 202311073048

(51) Int. Cl. A63B 24/00; A63B 23/00; A63B 71/06

(52) U.S. Cl. CPC A63B 24/0006; A63B 24/0062; A63B 24/0075; A63B 71/0619; A63B 2023/006; A63B 2024/0009; A63B 2024/0012; A63B 2024/0065; A63B 2024/0078; A63B 2071/0627; A63B 2071/0655; A63B 2208/0204; A63B 2214/00; A63B 2220/16; A63B 2220/62; A63B 2220/836; A63B 2225/54; A63B 2230/625

(57) ABSTRACT

A method for tracking and measuring body flexibility and balance of a user during physical activity is provided. The method includes capturing sensor data from a plurality of wearable devices for tracking and determining body posture, analyzing the captured sensor data to determine a plurality of parameters including information related to body posture and physical metrics including balance and flexibility, and displaying a balance score, a flexibility score, and transition over time based on the analysis.

### CROSS-REFERENCE TO RELATED APPLICATION(S)

This application is a continuation application, claiming priority under § 365 (c), of an International application No. PCT/KR2024/010835, filed on Jul. 25, 2024, which is based on and claims the benefit of an Indian Patent Application number 202311073048, filed on Oct. 26, 2023, in the Indian Intellectual Property Office, the disclosure of which is incorporated by reference herein in its entirety.

### TECHNICAL FIELD

The disclosure relates to physical fitness. More particularly, the disclosure relates to a system and method for tracking and measuring body flexibility and balance during physical activity.

### BACKGROUND ART

Physical activity is a fundamental aspect of maintaining a healthy lifestyle. It encompasses bodily movement produced by skeletal muscles that requires energy expenditure. There are several types of physical activities that are performed by an individual and yoga is a specific type of physical activity that has gained popularity worldwide. Yoga involves a combination of body postures, known as asanas, along with breathing exercise and is practiced by millions of people worldwide to achieve physical and mental well-being. While yoga offers many benefits, individuals practicing yoga may face challenges in performing the poses correctly and tracking their progress in terms of balance and flexibility over time. This can hinder their ability to fully reap the benefits of yoga practice.

Although there are various technologies that provide a yoga mode to measure metrics such as total time, average time, active time, heart rate ranges, and calories burnt, these technologies fail to provide an accurate assessment of correct yoga asanas, flexibility, and balance.

Therefore, there is a crucial need for a system or method that can accurately track and measure the body's flexibility and balance during the yoga.

Numerous prior art solutions exist that disclose about tracking the body posture of the user during the physical activity.

The related art discloses posture and motion monitoring using mobile devices. The related art further discloses a method that includes obtaining motion data from at least one motion sensor worn by a user, acquiring at least one frame of skeletal data of the user based on camera data or depth data, calibrating the motion data and skeletal data using at least one processor to determine a calibration offset, synchronizing the motion data and skeletal data and generating a body pose of the user with the at least one processor by aligning the reference frames of the synchronized motion data and synchronized skeletal data using the calibration offset, estimating the body pose of the user based on the aligned motion data and skeletal data and classifying the estimated body pose of the user using a machine learning model.

However, the related art does not disclose tracking and determining the body posture of the user during performance of the selected yoga asana using sensor data captured from wearable devices and providing real-time guidance for accurate execution. Further, the related art is silent about determining flexibility and balancing of the user using data captured from wearable devices during the yoga practice. In addition, the related art is silent about achieving transition of flexibility and balance from a beginner to an expert level based on the user's performance in the selected yoga asana.

Further, the related art discloses a yoga exercise body evaluation method and system. The prior art further discloses the yoga exercise method and the system are combined with the yoga style to evaluate body style of the user, and formulate a targeted yoga exercise scheme based on the user's body condition. The method includes obtaining yoga action posture information of a user and classifying it according to preset posture information for each evaluation dimension to generate dimension posture classification information, evaluating each yoga action posture of the user according to the dimension posture classification information and a preset body type evaluation standard for each evaluation dimension to generate body evaluation information for the user.

However, the related art does not disclose tracking and determining the body posture of the user during performance of the selected yoga asana using sensor data captured from wearable devices and providing real-time guidance for accurate execution. Further, the related art is silent about determining flexibility and balancing of the user using data captured from wearable devices during the yoga practice. In addition, the related art is silent about achieving transition of flexibility and balance from a beginner to an expert level based on the user's performance in the selected yoga asana.

Therefore, in light of the foregoing discussion, there exists a need to overcome the aforementioned drawbacks associated with the existing system and method for tracking and measuring body flexibility and balance of the user during physical activity.

The above information is presented as background information only to assist with an understanding of the disclosure. No determination has been made, and no assertion is made, as to whether any of the above might be applicable as prior art with regard to the disclosure.

### DISCLOSURE

Aspects of the disclosure are to address at least the above-mentioned problems and/or disadvantages and to provide at least the advantages described below. Accordingly, an aspect of the disclosure is to provide a system and method for tracking and measuring body flexibility and balance during physical activity.

Additional aspects will be set forth in part in the description which follows and, in part, will be apparent from the description, or may be learned by practice of the presented embodiments.

### Technical Solution

In accordance with an aspect of the disclosure, a method for tracking and measuring body flexibility and balance of a user during physical activity is provided. The method includes capturing sensor data from a plurality of wearable devices for tracking and determining body posture, analyzing the captured sensor data to determine a plurality of parameters including information related to body posture and physical metrics including balance and flexibility, and displaying a balance score, a flexibility score, and transition over time based on the analysis.

The method further includes analyzing the captured sensor data for determining a plurality of parameters including at least, but not limited to, information related to body posture and physical metrics such as balance and flexibility. In one embodiment, the analysis of the captured sensor data is performed for determining the plurality of parameters in steps, which include computing tilt angle using the sensor data captured from each of the wearable devices, combining all the computed tilt angles and mapping the combined tilt angle with tilt angle range of predefined poses of selected yoga asana from a database for determining user's current body posture, determining next body posture of the selected yoga asana by matching the determined current body posture with sequence of predefined postures from the database, and analyzing the sensor data for calculating the flexibility score that indicates degree of posture achieved by the user for the selected yoga asana and the balance score of current body posture and determining level of flexibility and balance of the body.

The balance score is calculated by determining variations in the sensor data and the level of flexibility and balance of the body includes beginner, intermediate and expert level. In an embodiment, the flexibility score and the balance score are stored in the database for future reference and guidance to the user. Thereafter, the steps include achieving transition of the level of flexibility and balance from beginner to expert level based on the user's performance in the selected yoga asana.

The transition of the level of flexibility and balance from the beginner to expert level is achieved by performing steps that includes recording duration of holding the posture of selected yoga asana and guiding the user to maintain the posture for a time duration based on personalized proficiency level of the user. The time duration guided to the user is calculated based on standard holding time of the posture for a yoga expert, previous time durations of holding the posture for the user, and current proficiency level of the user. In one embodiment, guidance is provided through wearable devices, such as audio cues and/or vibrations from the earbuds and vibrations from the smart watch.

The steps further include tracking the posture attained by the user while performing the selected yoga asana and guiding the user to change and attain the next posture correctly in sequence and providing real-time guidance to the user through audio to perform the posture accurately by analyzing and comparing the tilt angles computed for the user with stored data of the yoga expert. In one embodiment, an alarm is triggered in case the user attains a wrong posture.

In accordance with another aspect of the disclosure, a system for tracking and measuring body flexibility and balance of a user during physical activity is provided. The system includes a yoga pose input unit configured to enable the user to select a yoga asana as a physical activity, wherein the user selects the yoga asana using an electronic device, a sensor processing unit configured to capture sensor data from a plurality of wearable devices and compute a plurality of tilt angles, wherein the sensor data refers to plurality of motion sensor data, which is captured from wearable devices including a smart watch and earbuds, T a posture sensing unit configured to determine the user's current body posture and next body posture of the selected yoga asana, a physical metrics analytics unit configured to analyze the sensor data and calculate a flexibility score and a balance score of current body posture and determine a level of flexibility and balance of the body, and an instruction unit configured to achieve transition of the level of flexibility and balance from beginner to expert level based on the user's performance in the selected yoga asana.

In accordance with another aspect of the disclosure, one or more non-transitory computer-readable storage media storing one or more computer programs including computer-executable instructions that, when executed by one or more processors of an electronic device, cause the electronic device to perform operations are provided. The operations include capturing, during physical activity, sensor data from a plurality of wearable devices for tracking and determining body posture, analyzing the captured sensor data to determine a plurality of parameters including information related to body posture and physical metrics such as balance and flexibility, and displaying a balance score, a flexibility score, and transition over time based on the analysis.

Other aspects, advantages, and salient features of the disclosure will become apparent to those skilled in the art from the following detailed description, which, taken in conjunction with the annexed drawings, discloses various embodiments of the disclosure.

### DESCRIPTION OF DRAWINGS

The above and other aspects, features, and advantages of certain embodiments of the disclosure will be more apparent from the following description taken in conjunction the accompanying drawings, in which:

FIG. 1 depicts a flow diagram showing a method for tracking and measuring body flexibility and balance of a user during physical activity, according to an embodiment of the disclosure;

FIG. 2 depicts a flow diagram showing a method for analyzing captured sensor data to determine plurality of parameters, according to an embodiment of the disclosure;

FIG. 3 depicts a block diagram of the system performing method for tracking and measuring body flexibility and balance of the user during the physical activity, according to an embodiment of the disclosure;

FIG. 4 depicts a block diagram of a sensor processing module, according to an embodiment of the disclosure;

FIG. 5A depicts a block diagram of a posture sensing module, according to an embodiment of the disclosure;

FIG. 5B depicts a pictorial representation of sequence of poses of an Uttanasana yoga asana, according to an embodiment of the disclosure;

FIG. 6 depicts a flow diagram showing a method of working of the posture sensing module, according to an embodiment of the disclosure;

FIG. 7A depicts a block diagram of a physical metrics analytics module, according to an embodiment of the disclosure;

FIG. 7B depicts a pictorial representation of different levels of flexibility depending on user's proficiency level in performing the Uttanasana pose, according to an embodiment of the disclosure;

FIG. 7C depicts a pictorial representation of different graphs for different level of balance attained by the user, according to an embodiment of the disclosure;

FIG. 8A depicts a block diagram of an instruction module, according to an embodiment of the disclosure;

FIG. 8B depicts a pictorial representation of determining duration for holding the Uttanasana posture, according to an embodiment of the disclosure;

FIG. 8C depicts a pictorial representation of tracking and guiding correct posture to the user, according to an embodiment of the disclosure; and

FIG. 9 depicts a flow diagram showing a method of working of an instruction module, according to an embodiment of the disclosure.

The same reference numerals are used to represent the same elements throughout the drawings.

### MODE FOR INVENTION

The following description with reference to the accompanying drawings is provided to assist in a comprehensive understanding of various embodiments of the disclosure as defined by the claims and their equivalents. It includes various specific details to assist in that understanding, but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but are merely used by the inventor to enable a clear and consistent understanding of the disclosure. Accordingly, it should be apparent to those skilled in the art that the following description of various embodiments of the disclosure is provided for illustration purposes only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Furthermore, in the description, references to “one embodiment” or “an embodiment” means that a particular feature, structure, or characteristic described in connection with the embodiment is included in at least one embodiment of the disclosure. The appearance of the phrase “in one embodiment” in various places in the specification is not necessarily referring to the same embodiment, nor are separate or alternative embodiments mutually exclusive of other embodiments. Further, the terms “a” and “an” used herein do not denote a limitation of quantity, but rather denote the presence of at least one of the referenced items. Moreover, various features are described which may be exhibited by some embodiments and not by others. Similarly, various requirements are described, which may be requirements for some embodiments but not for other embodiments.

Yoga, an ancient practice that originated in India, has gained immense popularity worldwide for its numerous physical, mental, and spiritual benefits. With its roots dating back thousands of years, yoga provides a holistic approach to health and well-being.

One of the primary reasons people are drawn to yoga is its ability to provide physical fitness. Yoga encompasses various styles, each with its own unique focus and approach such as Uttanasana, UgraAsana, NagaAsana which are known asanas to improve flexibility, strength, and balance.

Furthermore, beyond the physical benefits, yoga also offers a myriad of mental and emotional benefits. The practice encourages mindfulness and self-awareness to reduce stress, anxiety, and depression. However, it is important to practice yoga correctly in order to fully enjoy the benefits of a healthier and more balanced life.

The disclosure aims to provide guidance on practicing yoga correctly and ensuring that individuals achieve maximum benefits by following proper techniques and aligning the body and breath.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display drive integrated circuit (IC), an audio CODEC chip, a universal serial bus (USB) controller, a camera controller, an image processing IC, a microprocessor unit (MPU), a system on chip (SoC), an integrated circuit (IC), or the like.

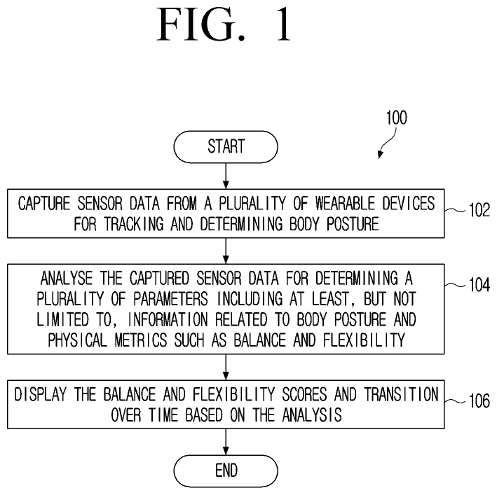

FIG. 1 depicts a flow diagram showing a method for tracking and measuring body flexibility and balance of a user during physical activity, according to an embodiment of the disclosure.

Referring to FIG. 1, a flow diagram showing a method (100) for tracking and measuring body flexibility and balance of a user during physical activity is disclosed. The physical activity refers to yoga asana and the tracking process includes guiding and correcting the user in real-time to ensure accurate performance of the yoga asana. In the flow diagram, each block may represent a module, segment, or portion of code, which comprises one or more executable instructions for implementing the specified logical function(s). It should also be noted that in certain alternative implementations, the sequence of functions depicted in the drawings may not necessarily occur in the exact order as indicated. For example, two blocks shown in succession in FIG. 1 may be executed substantially concurrently or the blocks may sometimes be executed in the reverse order, depending upon the functionality involved.

Any process descriptions or blocks in flowcharts should be understood as representing segments, modules, or portions of code that include one or more executable instructions for implementing specific logical functions or steps in the process, and alternate implementations are included within the scope of the example embodiments in which functions may be executed out of order from that shown or discussed, including substantially concurrently or in reverse order, depending on the functionality involved. In addition, the process descriptions or blocks in the flow charts should be understood as representing decisions made by a hardware structure such as a state machine. The flow diagram starts at operation 102 and proceeds to operation 106.

At operation 102, sensor data is captured from a plurality of wearable devices for tracking and determining body posture. The sensor data may be plurality of motion sensor data, which is captured from wearable devices such as a smart watch and earbuds. The plurality of motion sensor data may be collected from multiple types of motion sensors, including at least but not limited to accelerometers, gyroscopes, and magnetometers, which are integrated into the wearable devices to capture and measure movement.

It is important to note that accelerometers are designed to measure acceleration, which is the rate of change of velocity per unit of time. The accelerometers are capable of detecting both linear acceleration i.e. movement in a straight line and gravitational acceleration i.e. the force that attracts a body toward the center of the Earth for providing valuable information about motion, orientation, and physical activity of the user.

The gyroscopes are integrated to track and measure angular velocity, rotation, and orientation. The gyroscopes generally provide information about the device's angular position and angular movement in three-dimensional space.

The magnetometers are designed to detect and measure changes in magnetic fields. They work in conjunction with other sensors, such as accelerometers and gyroscopes, to track and determine body posture. By combining data from multiple motion sensors, it is possible to obtain a more comprehensive understanding of motion patterns and behaviors of the user's body.

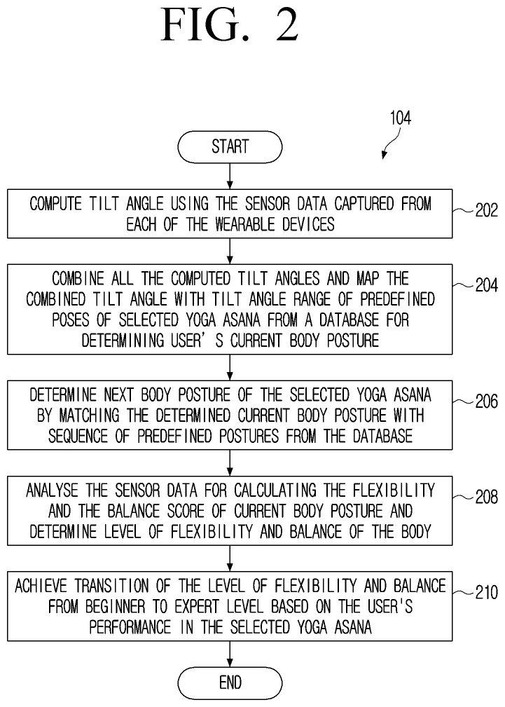

Successively, the captured sensor data is analyzed, at operation 104, for determining a plurality of parameters including at least, but not limited to, information related to body posture and physical metrics such as balance and flexibility. The analysis of the captured sensor data may be performed in a series of operations, as shown in FIG. 2.

FIG. 2 depicts a flow diagram showing a method for analyzing the captured sensor data to determine the plurality of parameters, according to an embodiment of the disclosure.

Referring to FIG. 2, the analysis method includes computing tilt angle using the sensor data captured from each of the wearable devices, at operation 202. The tilt angle refers to the angle at which the wearable device is tilted or inclined with respect to a reference plane. It is important to note that computation of the tilt angle involves analyzing the plurality of motion sensor data from the motion sensors to determine the orientation of the wearable devices in relation to the Earth's gravitational field.

The accelerometer sensor that is one of the motion sensors in the wearable devices measure acceleration, including gravitational acceleration, which allows for the calculation of the tilt angle. By analyzing the acceleration data along different axes typically in x, y, and z axes, the orientation of the wearable device is determined.

The analysis method further includes combining all the computed tilt angles and mapping the combined tilt angle with tilt angle range of predefined poses of selected yoga asana, at operation 204, for determining user's current body posture. In one embodiment these predefined poses and their corresponding tilt angle ranges may be stored in a database.

The analysis method further includes determining next body posture of the selected yoga asana, at operation 206. The next body posture is determined by matching the determined current body posture with sequence of predefined postures from the database.

The analysis method further includes analyzing the sensor data for calculating the flexibility and the balance score of current body posture and determining level of flexibility and balance of the body, at operation 208. The sensor data may be analyzed to calculate flexibility and balance score of current body posture and to determine level of flexibility and balance of the body. The flexibility score indicates degree of posture achieved by the user for the selected yoga asana. It should be noted that the flexibility refers to range of motion available at a joint or a group of joints. It is the ability of muscles and joints to move through their full range without discomfort or restriction. Good flexibility allows for smooth and unrestricted movement, enhances performance in physical activities, and reduces the risk of injuries. The level of flexibility includes beginner, intermediate and expert level.

The balance score is calculated by determining variations in the sensor data. It should be noted that the balance refers to ability to maintain control and stability of the body's position, whether it is stationary or in motion. It involves the coordination of multiple systems, including the musculoskeletal, sensory, and nervous systems. Good balance is crucial for yoga, especially for poses that require stability and maintaining postural alignment. The level of balance includes various levels such as beginner, intermediate, and expert. A lower balance score or higher variations in the score indicate user instability, placing them in the beginner level of balance. Conversely, higher balance scores indicate better stability and control, placing individuals at a more advanced level of balance, such as intermediate or expert. The flexibility score and the balance score are stored in the database for future reference and guidance to the user.

Furthermore, the analysis method includes achieving transition of the level of flexibility and balance from beginner to expert level, at operation 210, based on the user's performance in the selected yoga asana. The transition may be facilitated by providing personalized guidance and real-time feedback through wearable devices to help users maintain correct postures and improve their proficiency over time.

Referring back to FIG. 1, the balance and flexibility scores and transition determined over time based on the analysis are displayed, at operation 106.

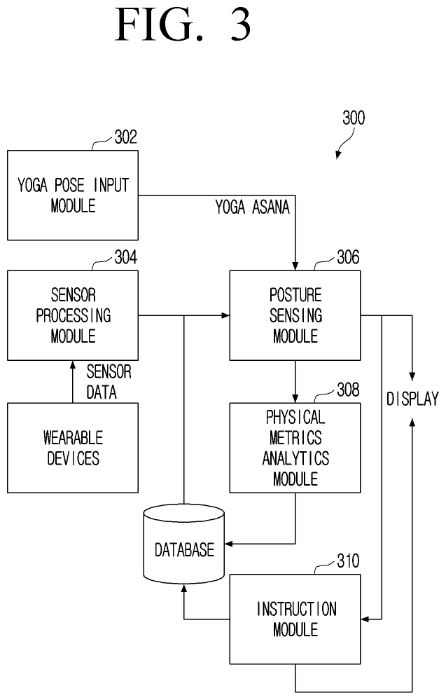

FIG. 3 depicts a block diagram of the system performing method for tracking and measuring body flexibility and balance of the user during the physical activity, according to an embodiment of the disclosure.

Referring to FIG. 3, the physical activity refers to yoga asana, which includes a variety of poses or postures. Some examples of these poses, but not limited to, are Uttanasana (standing forward bend), Ardhachandrasana (half-moon pose), Parsvasukhasana (seated side stretch), Ugrasana (fierce pose), and Nagaasana (cobra pose). Yoga asanas, also referred to as yoga poses or yoga postures, are physical postures that are practiced as a fundamental component of the broader discipline of yoga.

As depicted, the system 300 comprises a yoga pose input module 302 which is configured for enabling the user to select a yoga asana, wherein the user selects the yoga asana using an electronic device. According to various embodiments of the disclosure, the electronic device may include a mobile phone, computer, laptop, notebook, and any other device which includes a processing module and a user interface for enabling the user to select the yoga asana. The electronic device is also capable of connecting to the wearable devices through any communication means including at least, but not limited to, Bluetooth.

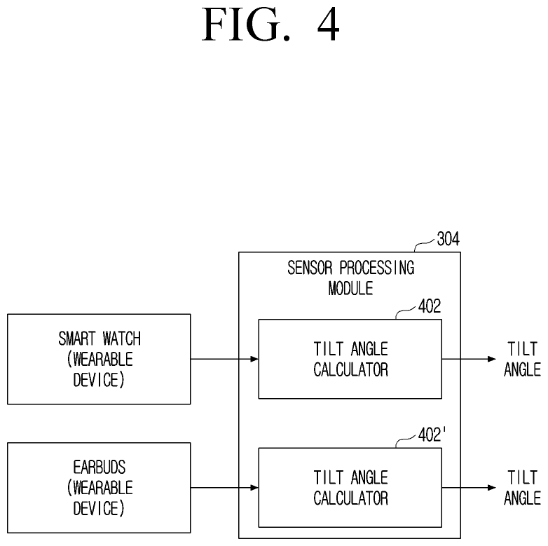

The system 300 further comprises a sensor processing module 304 for capturing sensor data from a plurality of wearable devices and computing tilt angle. In one embodiment, the sensor data refers to a plurality of motion sensor data, which is captured from the wearable devices such as a smart watch and earbuds. The sensor processing module 304 is described below with respect to FIG. 4.

FIG. 4 depicts a block diagram of the sensor processing module (304), according to an embodiment of the disclosure.

Referring to FIG. 4, the sensor processing module 304 comprises a plurality of tilt angle calculators 402 and 402′. Each tilt angle calculator 402 and 402′ is configured to receive the sensor data from each wearable device and provide tilt angles at the output. These angles are then used for posture recognition of the selected yoga asana in subsequent steps. It should be noted that each tilt angle calculator computes tilt angle using sensor data received from smart watch and earbuds. In one embodiment, the tilt angles are computed as the angle of inclination of the device sensor axes with reference to the ground surface using equations 1 and 2 below:

$$X angle = \frac{180}{π} * {sin}^{- 1} ( \frac{{a}_{x}}{\sqrt{{a}_{x}^{2} + {a}_{y}^{2} + {a}_{z}^{2}}} ) \qquad Equation 1$$

$$Y angle = \frac{180}{π} * {sin}^{- 1} ( \frac{{a}_{y}}{\sqrt{{a}_{x}^{2} + {a}_{y}^{2} + {a}_{z}^{2}}} ) \qquad Equation 2$$

$$Z angle = ( ( \frac{180}{π} * {cos}^{- 1} ( \frac{{a}_{z}}{\sqrt{{a}_{x}^{2} + {a}_{y}^{2} + {a}_{z}^{2}}} ) ) - 90 ) * - 1 \qquad Equation 3$$

In Equations 1 and 2, aₓ, a_{y}, and a_{z} are the acceleration data along x, y, and z axis.

In another embodiment, the tilt angles may be computed using the sensor data by utilizing other than above mentioned equation.

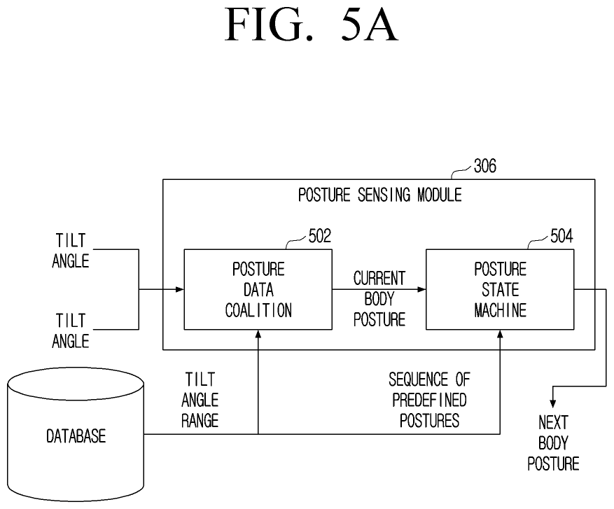

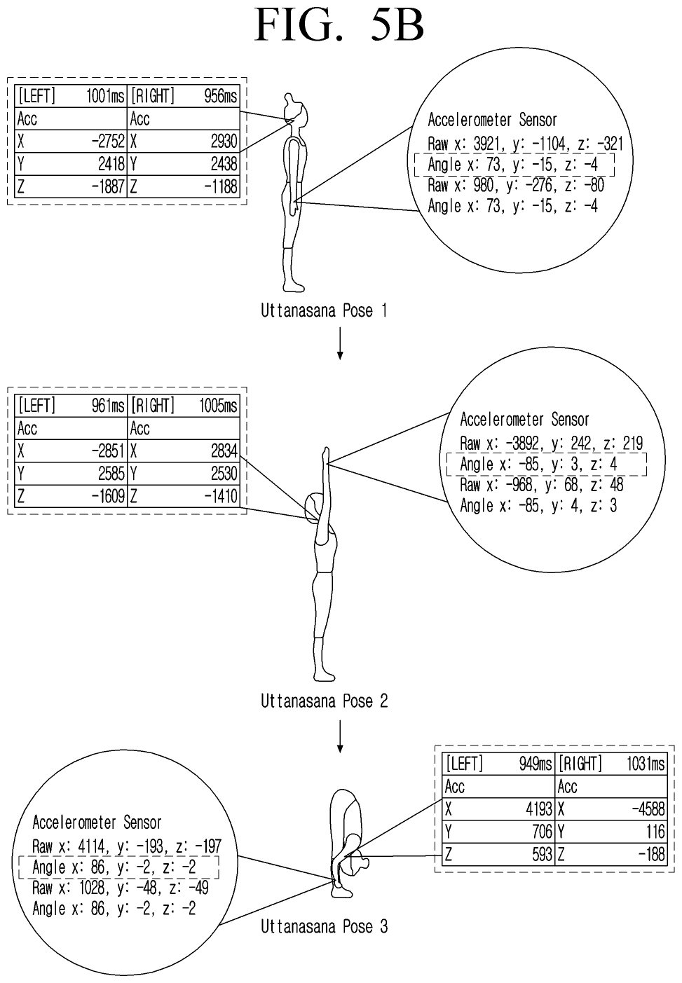

Referring back to FIG. 3, the system 300 further comprises a posture sensing module 306 for determining user's current body posture and next body posture of the selected yoga asana, which is described below with respect to FIGS. 5A and 5B.

FIG. 5A depicts a block diagram of the posture sensing module is depicted, according to an embodiment of the disclosure.

Referring to FIG. 5A, the posture sensing module 306 includes a posture data coalition 502. The posture data coalition 502 may be configured to combine all the tilt angles computed by the sensor processing module 304 using sensor data captured from the smart watch and the earbuds. The posture data coalition 502 is further configured to map the combined tilt angle with tilt angle range of predefined poses of selected yoga asana from a database for determining user's current body posture. This mapping process helps determine the user's current body posture during the yoga asana.

FIG. 5B depicts a pictorial representation of sequence of poses of the Uttanasana yoga asana, according to an embodiment of the disclosure.

Referring to FIG. 5B, each pose has a specific body posture that is determined from the sensor data received from the earbuds and smart watch worn by the user. The sensors in these devices capture relevant data such as tilt angles, movement patterns, and other parameters that are then combined and mapped with tilt angle range of predefined poses to accurately determine the user's body posture during each pose.

It is important to note that the database contains the tilt angle range for all the predefined poses of all the yoga asanas. For example, in case of the Uttanasana yoga asana, the database contains angle ranges, which is disclosed in Table 1 as shown below:

TABLE 1
Angle ranges for the smart watch and earbuds for
different postures of the Uttanasana yoga asana

| Posture | Smart watch Angle Range | Earbuds Angle Range |
|---|---|---|
| Pose 1 | {70:90, −10:10, −10:10} | {15:25, 50:70, −30:−10} |
| | | {−25:−15, 70:78, −20:0} |
| Pose 2 | {−70:−50, −10:10, 15:35} | {15:25, 50:70, −30:−10} |
| | | {−25:−15, 70:78, −20:0} |
| Pose 3 | {70:85, −10:10, −5:−15} | {70:90, −10:10, −15:10} |
| | | {−90:−70, −10:10, −5:15} |

As disclosed, for pose 1, the angle range for the smart watch should be 70 to 90 degrees, −10 to 10 degrees, and −10 to 10 degrees. The angle range for one of the earbuds, either left or right, should be 15 to 25 degrees, 50 to 70 degrees, and −30 to −10 degrees and for the other earbud, the angle range should be −25 to −15 degrees, 70 to 78 degrees, and −20 to 0 degrees. Similarly, the angle ranges for other poses are also stored in the database.

The posture sensing module 306 further includes a posture state machine 504. The posture state machine 504 is configured for determining next body posture of the selected yoga asana. In one embodiment, the next body posture is determined by matching the determined current body posture, received from the posture data coalition 502, with a sequence of predefined postures or poses stored in the database. In addition current posture stay time is extracted from the database based on the determined current body posture for guiding the next posture of the selected yoga asana.

The database may also contain a sequence of poses for different yoga asana and information disclosing pose time, flexibility and balance weight for each pose of the yoga asana. In an embodiment, Tables 2 and 3 discloses information about Uttanasana yoga asana as shown below:

TABLE 2
Posture sequences for different yoga asana

| Yoga asana Name | Posture Sequence |
|---|---|
| Uttanasana | Pose1, Pose2, Pose3, . . . |
| Ugraasana | Pose1, Pose2, Pose3, . . . |
| Nagaasana | Pose1, Pose2, Pose3, . . . |

As shown in Table 2, different yoga asanas such as, but not limited to, Uttanasana, Ugraasana, and Nagaasana and their respective poses are stored in the database.

TABLE 3
Specific information of Uttanasana yoga asana

| Posture | Posture Time | Flexibility Weight | Balance Weight |
|---|---|---|---|
| Pose 1 | 2 | 0 | 0.1 |
| Pose 2 | 3 | 0.1 | 0.2 |
| Pose 3 | 30 | 0.8 | 0.3 |

As shown in Table 3, specific information such as posture time, flexibility and balance weights related to each pose of the yoga asana is stored in the database.

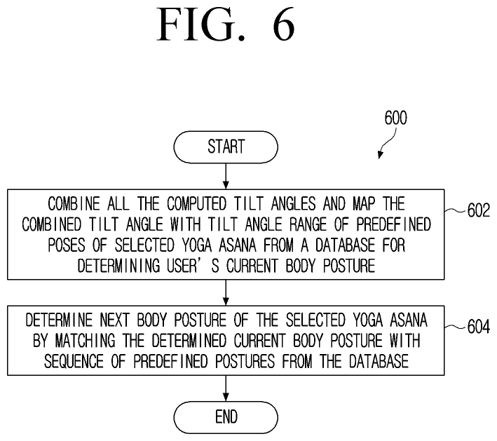

FIG. 6 depicts a flow diagram 600 showing a method of working of the posture sensing module, according to an embodiment of the disclosure.

Referring to FIG. 6, the method includes combining all the tilt angles computed using sensor data captured from the smart watch and the earbuds and mapping the combined tilt angle with tilt angle range of predefined poses of selected yoga asana from a database for determining user's current body posture, at operation 602.

Thereafter, the method includes determining next body posture of the selected yoga asana by matching the determined current body posture with sequence of predefined postures from the database, at operation 604.

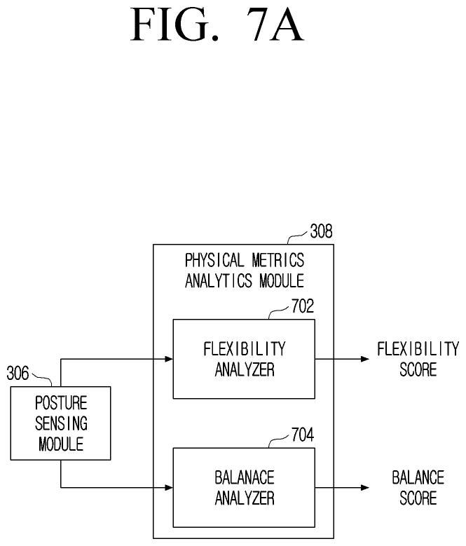

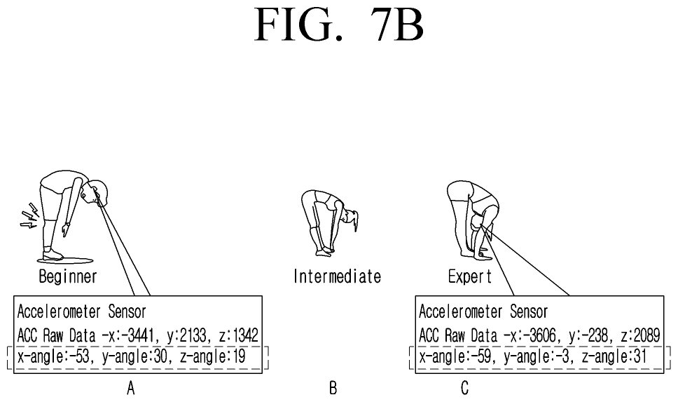

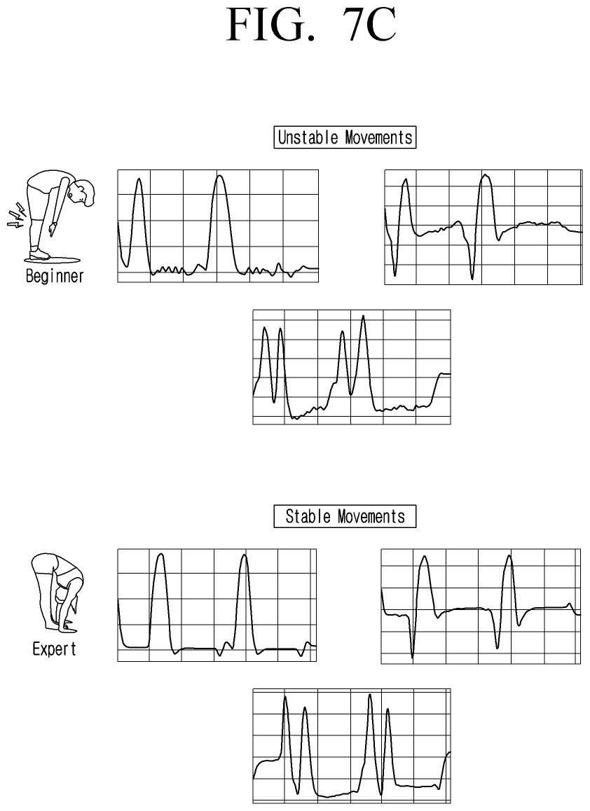

The system 300 further comprises a physical metrics analytics module 308, which is configured for analyzing the sensor data and calculating the flexibility and the balance score of current body posture and determining level of flexibility and balance of the body. According to an embodiment described below with respect to FIG. 7A, the physical metrics analytics module 308 may include a flexibility analyzer 702 and a balance analyzer 704.

FIG. 7A depicts a block diagram of the physical metrics analytics module 308, according to an embodiment of the disclosure.

Referring to FIG. 7A, the flexibility analyzer 702 and balance analyzer 704 receive input from the posture sensing module 306 and compute flexibility and balance scores of the current body posture.

The flexibility analyzer 702 assesses the extent to which the current body posture exhibits flexibility by analyzing factors such as joint mobility, muscle length, and range of motion and uses this information to calculate a flexibility score. The flexibility score indicates how well the user is able to achieve and maintain the current body posture. The flexibility analyzer 702 further determines the level of flexibility which includes beginner, intermediate and expert level, as depicted in FIG. 7B.

FIG. 7B depicts a pictorial representation of different levels of flexibility, according to an embodiment of the disclosure. The sensor data differs depending on the user's proficiency level in performing the Uttanasana pose.

Referring to part “A” of FIG. 7B, the user is categorized as a beginner since the sensor data indicates that the user's hands or fingertips are not reaching the ground while maintaining a straight knee position. This categorization is based on the requirement of touching the ground which the user is not able to achieve in this pose. This level suggests that the user is working on developing the flexibility and range of motion required for the pose.

Referring to part “B” of FIG. 7B, the user is categorized as intermediate, since the user is able to touch their feet but not the ground while maintaining the appropriate body alignment. This indicates that the user has made progress in terms of flexibility and is able to achieve a deeper fold in the pose.

Referring to part “C” of FIG. 7B, the user is categorized as an expert who performs the asana properly, the sensor data indicates that the user is able to touch the ground comfortably while maintaining a straight knee position. This data reflects the user's advanced level of flexibility and mastery of the pose.

The balance analyzer 704 analyzes the sensor data captured from the wearable devices to calculate the balance score of the current body posture. In one embodiment, the balance analyzer 704 calculates the balance and stability of the current body posture by determining variations in the sensor data using the equation 4 given below:

$${Variance}_{X} = {\sum }_{i}^{n} {( {X}_{i} - {Mean}_{X} )}^{2} / n \qquad Equation 4$$

In Equation 4, X represents observation or value of the sensor data at a particular instant, n represents the total number of observations.

It should be noted that the balance score indicates how well the user is able to maintain equilibrium and control during the posture.

The balance analyzer 704 further determines level of balance which includes beginner, intermediate, and expert levels.

FIG. 7C shows a pictorial representation of different graphs depicting the level of balance attained by the user, according to an embodiment of the disclosure.

Referring to FIG. 7C, when the user is a beginner, the graphs show unstable movements. Conversely, when the user is an expert, the graphs show stable movements.

By continuously analyzing the input from the posture sensing module, the flexibility analyzer and balance analyzer provide scores on the user's flexibility and balance in real-time, which are then stored in the database for future reference and further guidance to the user.

According to another embodiment of the disclosure, the balance and stability of the current body posture may be calculated by analyzing factors such as weight distribution, alignment, and control.

Table 4 discloses different examples of user personalized data including flexibility and balance scores for the user with respect to different body postures.

TABLE 4
Flexibility and balance scores for the user
with respect to different body postures

| Posture | Posture Time (S) | Flexibility Score | Balance Score |
|---|---|---|---|
| Pose 1 | 2 | 10 | 10 |
| Pose 2 | 3 | 8 | 8 |
| Pose 3 | 18 | 7 | 8 |

As disclosed, when the user is in pose 1 for time duration of 2 seconds, the physical metrics analytics module 308 determines the flexibility score of 10 and the balance score of 10. This indicates a high level of flexibility and good balance of the user in the pose 1.

Similarly, when the user is in pose 2 for time duration of 3 seconds, the physical metrics analytics module determines the flexibility score of 8 and the balance score of 8. This indicates a slightly lower flexibility score and balance score compared to pose 1.

In the case of pose 3, when the user maintains the pose for time duration of 18 seconds, the physical metrics analytics module determines the flexibility score of 7 and the balance score of 8. This indicates a slightly lower flexibility score, but the balance score remains consistent with pose 2. It suggests that the user may need to work on improving their flexibility in pose 3 while maintaining good balance.

According to an embodiment of the disclosure, the physical metrics analytics module 308 may further display the balance and flexibility scores to the user through the wearable device or the electronic device.

Furthermore, the physical metrics analytics module 308 further provides a level based on the level of flexibility and balance as shown in Table 5 below:

TABLE 5
level based on the level of flexibility and balance

| Level of Flexibility | Level of Balance | Level |
|---|---|---|
| Beginner | Beginner | Beginner |
| Intermediate | Beginner | |
| Expert | Beginner | Intermediate |
| Intermediate | Intermediate | |
| Expert | Intermediate | Expert |
| Expert | Expert | |

As shown in Table 5, if both flexibility and balance are determined as beginner or if one level is intermediate and another is determined as beginner, the level may be beginner. If either both balance and flexibility are determined as intermediate or if one level is expert and another is determined as beginner, the level may be intermediate. Finally, if both balance and flexibility are determined as expert or if one level is expert and another is determined as intermediate, the level may be expert.

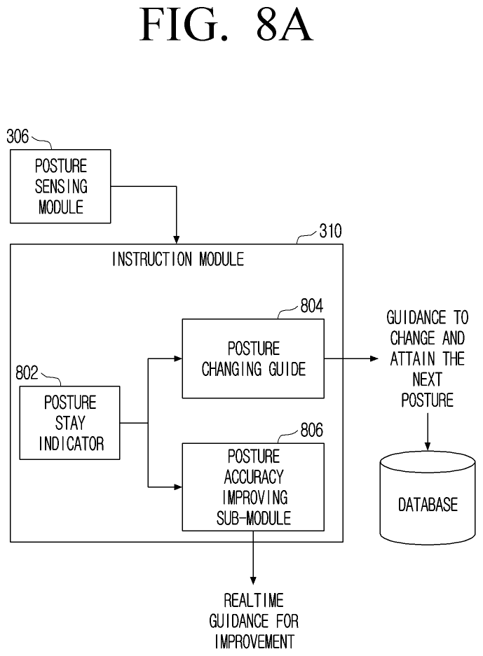

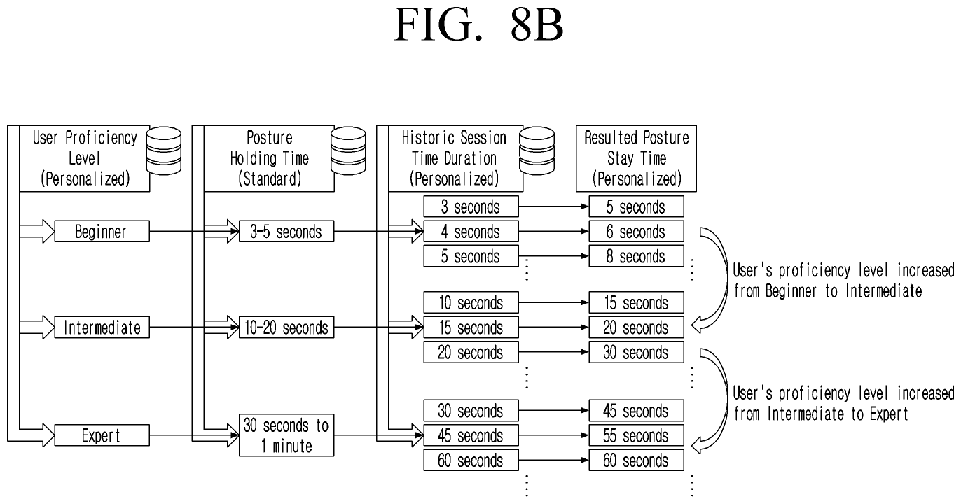

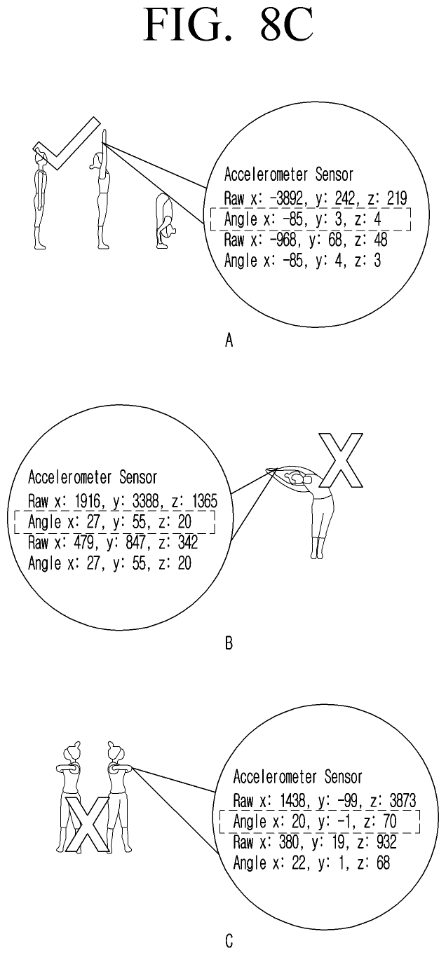

The system further comprises an instruction module 310, which is configured to achieve transition of the level of flexibility and balance from beginner to expert level based on the user's performance in the selected yoga asana and display the transition of the level over time to the user through the wearable device or the electronic device. The instruction module 310 is described below with respect to FIG. 8A.

FIG. 8A depicts a block diagram of the instruction module is depicted, according to an embodiment of the disclosure.

Referring to FIG. 8A, the instruction module 310 includes a posture stay indicator 802 for recording duration of holding the posture of selected yoga asana. The posture stay indicator 802 calculates the time duration guided to the user based on standard holding time of the posture for a yoga expert, previous time durations of holding the posture for the user, and current proficiency level of the user, which is described below with respect to FIG. 8B.

FIG. 8B depicts a pictorial representation of determining the duration for holding the Uttanasana posture, according to an embodiment of the disclosure.

Referring to FIG. 8B, the recommended posture holding time is personalized for each user based on standard holding time of the posture, previous time durations of holding the posture for the user, and current proficiency level of the user.

For instance, the beginner user may be guided to stay in pose 1, pose 2, and pose 3 of the Uttanasana for about 5 seconds, 6 seconds, and 8 seconds respectively depending upon the standard holding time of the posture 3-5 and previous time durations of holding the posture for the user 3 seconds, 4 seconds, and 5 seconds.

Intermediate user may be guided to stay in pose 1, pose 2, and pose 3 of the Uttanasana for duration of 15 seconds, 20 seconds, and 30 seconds respectively and while expert may be guided to comfortably hold the posture for 45 seconds, 55 seconds, and 60 seconds.

These determinations may be calculated based on historic time duration of the user for holding the posture, standard holding time and proficiency level of the user. By tailoring the duration to each individual, it allows for a more customized and effective practice of the yoga posture. In one embodiment, the guidance is provided through wearable devices, such as audio cues and/or vibrations from the earbuds and vibrations from the smart watch.

The instruction module 310 further includes a posture changing guide 804 configured for tracking the posture attained by the user while performing the selected yoga asana and guiding the user to change and attain the next posture correctly in sequence, as depicted in FIG. 8C.

FIG. 8C depicts a pictorial representation of tracking and guiding correct posture to the user, according to an embodiment of the disclosure.

Referring to part “A” of FIG. 8C, which depicts the posture attained by the user is correct according to the stored the standard sequence of postures sensor data. However, parts “B” and “C” of FIG. 8C depict postures attained by the user are incorrect and do not align with the stored standard posture of the yoga asana. The posture changing guide 804 is further configured for triggering an alarm in case of wrong posture attained by the user. It should be noted that every yoga asana has a predefined sequence of steps that needs to be followed.

The posture changing guide 804 is configured to store the standard sequence of postures and guides the user through the complete yoga asana, ensuring all the postures are achieved correctly and in the correct sequence. The correct sequence of steps is stored in terms of a range of inclination angles gathered from data of the yoga experts. If the user follows the wrong steps, the posture changing guide (804) compares the inclination angles achieved by the user with the predefined range of angle values. If the user's angles deviate from the correct range, an audio feedback through earbuds and haptic feedback through a smart watch to help the user achieve the correct posture.

The instruction module 310 further includes a posture accuracy improving sub-module 806 for analyzing and comparing the tilt angles computed for the user with stored data of the yoga expert. The posture accuracy improving sub-module 806 aims to improve the user's flexibility by providing real-time guidance through audio cues to assist the user in enhancing their flexibility. The posture accuracy improving sub-module 806 may guide the user to stretch a little further in order to achieve a more precise pose.

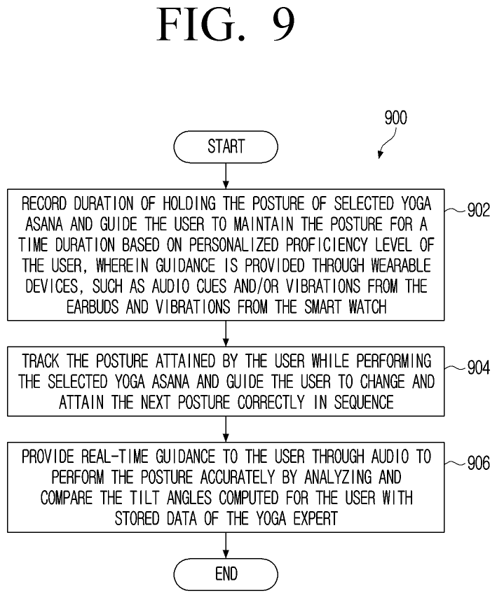

FIG. 9 depicts a flow diagram showing a method of working of the instruction module (310) for transition of the level of flexibility and balance from the beginner to expert level, according to an embodiment of the disclosure.

Referring to FIG. 9, the method 900 includes recording duration of holding the posture of selected yoga asana and guiding the user to maintain the posture for time duration, at operation 902, based on personalized proficiency level of the user. The time duration is calculated based on standard holding time of the posture for the yoga expert, previous time durations of holding the posture for the user, and current proficiency level of the user. In one embodiment, guidance is provided through wearable devices, such as audio cues and/or vibrations. For example, audio cues may be delivered through earbuds, providing verbal instructions or reminders to the user. Vibrations from a smart watch may also be used to provide tactile feedback and cues for maintaining the posture correctly.

The method further includes tracking the posture attained by the user while performing the selected yoga asana and guiding the user to change and attain the next posture correctly in sequence, at operation 904. It should be noted that the tracking of the posture attained by the user may involve analyzing the position of various body parts, such as the arms, legs, hands, and spine, to ensure proper alignment and form. In one embodiment, an alarm is triggered in case the user attains a wrong posture. The alarm serves as a feedback mechanism to alert the user that their posture is incorrect and needs adjustment.

Thereafter, the method includes providing real-time guidance to the user through audio to perform the posture accurately by analyzing and comparing the tilt angles computed for the user with stored data of the yoga expert, at operation 906. This guidance helps the user to make necessary adjustments and achieve the correct tilt angles and alignment to perform the yoga asana with improved accuracy.

It has thus been seen that the system and method for tracking and measuring body flexibility and balance of the user during physical activity according to the disclosure achieve the purposes highlighted earlier. Such a system and method can in any case undergo numerous modifications and variants, all of which are covered by the same innovative concept, moreover, all of the details can be replaced by technically equivalent elements.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device individually or collectively, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A method for tracking and measuring body flexibility and balance of a user during physical activity, the method comprising:
capturing sensor data from a plurality of wearable devices for tracking and determining body posture;
analyzing the captured sensor data to determine a plurality of parameters including information related to body posture and physical metrics including balance and flexibility; and
displaying a balance score, a flexibility score, and transition over time based on the analysis.

2. The method as claimed in claim 1, wherein the physical activity refers to yoga asana and a tracking process includes guiding and correcting the user in real-time to ensure accurate performance of the yoga asana.

3. The method as claimed in claim 1, wherein the sensor data refers to a plurality of motion sensor data, which is captured from the wearable devices such as a smart watch and earbuds.

4. The method as claimed in claim 1, wherein the analysis of the captured sensor data is performed for determining the plurality of parameters in steps, which comprises:
computing a plurality of tilt angles using the sensor data captured from each of the wearable devices;
combining the computed plurality of tilt angles and mapping the combined tilt angle with a tilt angle range of predefined poses of selected yoga asana from a database for determining user's current body posture;
determining a next body posture of the selected yoga asana by matching the determined current body posture with sequence of predefined postures from the database;
analyzing the sensor data for calculating the flexibility and a balance score of current body posture and determining level of flexibility and balance of the body; and
achieving transition of the level of flexibility and balance from beginner to expert level based on the user's performance in the selected yoga asana.

5. The method as claimed in claim 4, wherein the flexibility score and the balance score are stored in the database for future reference and guidance to the user.

6. The method as claimed in claim 4, wherein the flexibility score indicates degree of posture achieved by the user for the selected yoga asana, the level of flexibility includes beginner, intermediate and expert level.

7. The method as claimed in claim 4, wherein the balance score is calculated by determining variations in the sensor data and the level of balance includes beginner, intermediate and expert level, where higher variations indicate user instability.

8. The method as claimed in claim 4, wherein the transition of the level of flexibility and balance from the beginner to expert level is achieved by performing steps comprising:
recording duration of holding the posture of selected yoga asana and guiding the user to maintain the posture for a time duration based on personalized proficiency level of the user, wherein guidance is provided through wearable devices, such as audio cues and/or vibrations from earbuds and vibrations from a smart watch;
tracking the posture attained by the user while performing the selected yoga asana and guiding the user to change and attain the next posture correctly in sequence; and
providing real-time guidance to the user through audio to perform the posture accurately by analyzing and comparing the tilt angles computed for the user with stored data of a yoga expert.

9. The method as claimed in claim 8, wherein the time duration guided to the user is calculated based on standard holding time of the posture for a yoga expert, previous time durations of holding the posture for the user, and current proficiency level of the user.

10. The method as claimed in claim 8, wherein an alarm is triggered in case the user attains a wrong posture.

11. A system for tracking and measuring body flexibility and balance of a user during physical activity, the system comprising:
a yoga pose input unit configured to enable the user to select a yoga asana as a physical activity, wherein the user selects the yoga asana using an electronic device;
a sensor processing unit configured to capture sensor data from a plurality of wearable devices and compute a plurality of tilt angles, wherein the sensor data refers to a plurality of motion sensor data, which is captured from the wearable devices including a smart watch and earbuds;
a posture sensing unit configured to determine the user's current body posture and next body posture of the selected yoga asana;
a physical metrics analytics unit configured to analyze the sensor data and calculate a flexibility score and a balance score of current body posture and determine a level of flexibility and balance of the body; and
an instruction unit configured to achieve transition of the level of flexibility and balance from beginner to expert level based on the user's performance in the selected yoga asana.

12. The system as claimed in claim 11,
wherein the system is further configured to display the balance and flexibility scores and transition over time to the user through a wearable device or an electronic device, and
wherein the electronic device includes a mobile phone.

13. The system as claimed in claim 11, wherein the posture sensing unit comprises:
a posture data coalition configured to combine the computed plurality of tilt angles com and map the combined tilt angle with a tilt angle range of predefined poses of selected yoga asana from a database for determining user's current body posture; and
a posture state machine configured to determine a next body posture of the selected yoga asana by matching the determined current body posture with sequence of predefined postures from the database.

14. The system as claimed in claim 11, wherein the physical metrics analytics unit comprises:
a flexibility analyzer configured to analyze the sensor data captured from the wearable devices to calculate the flexibility score of current body posture, wherein the flexibility score indicates degree of posture achieved by the user for the selected yoga asana and is used for determining the level of flexibility which includes beginner, intermediate and expert level; and
a balance analyzer configured to analyze the sensor data captured from the wearable devices to calculate the balance score of current body posture, wherein the balance score is calculated by determining variations in the sensor data and the level of balance includes beginner, intermediate and expert level.

15. The system as claimed in claim 11, wherein the instruction unit comprises:
a posture stay indicator configured to record a duration of holding the posture of selected yoga asana and guide the user to maintain the posture for a time duration based on personalized proficiency level of the user, wherein guidance is provided through wearable devices, such as audio cues and/or vibrations from the earbuds and vibrations from the smart watch;
a posture changing guide configured to track the posture attained by the user while performing the selected yoga asana and guide the user to change and attain the next posture correctly in sequence; and
a posture accuracy improving unit configured to analyze and compare the tilt angles computed for the user with stored data of a yoga expert.

16. The system as claimed in claim 11, further comprising:
memory storing a database,
wherein the flexibility score and the balance score are stored in the database for future reference and guidance to the user.

17. The system as claimed in claim 11,
wherein the balance score is one of beginner, intermediate, and expert, and
wherein the balance score is determined based on variations in the sensor data.

18. The system as claimed in claim 11,
wherein the sensor processing unit further comprises a plurality of tilt angle calculators, each of which correspond to one of the plurality of wearable devices, and
wherein each of the plurality of tilt angle calculators are configured to generate a tilt angle based on sensor data from the corresponding wearable device.

19. One or more non-transitory computer-readable storage media storing one or more computer programs including computer-executable instructions that, when executed by one or more processors of an electronic device, cause the electronic device to perform operations, the operations comprising:
capturing, during physical activity, sensor data from a plurality of wearable devices for tracking and determining body posture;
analyzing the captured sensor data to determine a plurality of parameters including information related to body posture and physical metrics such as balance and flexibility; and
displaying a balance score, a flexibility score, and transition over time based on the analysis.

20. The one or more non-transitory computer-readable storage media of claim 19, wherein the physical activity refers to yoga asana and a tracking process includes guiding and correcting a user in real-time to ensure accurate performance of the yoga asana.
